(19) United States
(12) Patent Application Publication
(10) Pub. No.: US 2024/0399205 A1
(43) Pub. Date: Dec. 5, 2024

(54) TECHNIQUES FOR MANAGING WORKOUT PROFILES ON COMPUTING DEVICES

(71) Applicant: Apple Inc., Cupertino, CA (US)

(72) Inventors: Michael D. FORD, Portola Valley, CA (US); Tu K. NGUYEN, Fountain Valley, CA (US)

(21) Appl. No.: 18/505,087

(22) Filed: Nov. 8, 2023

Related U.S. Application Data
(60) Provisional application No. 63/506,074, filed on Jun. 3, 2023.

(51) Int. Cl. A63B 24/00; A63B 71/06

(52) U.S. Cl. CPC A63B 24/0062; A63B 24/0075; A63B 71/0622; A63B 2024/0065; A63B 2024/0078

(57) ABSTRACT

The embodiments set forth techniques for managing workout profiles on computing devices. In particular, the techniques include a method that is implemented by a fitness application executing on a client device, and include the steps of (1) receiving a workout object from an entity that is external to the fitness application, (2) generating, within the fitness application, a workout profile that is based on the workout object, where the workout profile is associated with at least one condition, (3) monitoring information gathered from at least one sensor that is communicatively coupled to the client device, and (4) in response to determining, based on the information, that the at least one condition is satisfied: causing at least one user interface to reflect that the at least one condition is satisfied. The techniques also include a method for generating workout objects from which workout profiles can be derived on client devices.

### CROSS-REFERENCE TO RELATED APPLICATIONS

The present application claims the benefit of U.S. Provisional Application No. 63/506,074, entitled “TECHNIQUES FOR MANAGING WORKOUT PROFILES ON COMPUTING DEVICES,” filed Jun. 3, 2023, the content of which is incorporated by reference herein in its entirety for all purposes.

### FIELD

The described embodiments relate generally to techniques for managing workout profiles on computing devices. More particularly, the described embodiments provide techniques for generating workout objects from which workout profiles can be derived on client devices. The described embodiments also provide techniques for deriving workout profiles from workout objects on client devices.

### BACKGROUND

Fitness trackers, paired with innovative software, have revolutionized the way individuals monitor and enhance their physical well-being. These devices, typically worn on the wrist like a watch, have become immensely popular due to their ability to track various aspects of fitness and provide valuable insights into one's daily activities. With the help of advanced sensors, fitness trackers can monitor metrics such as steps taken, distance covered, calories burned, heart rate, sleep patterns, and even stress levels. This data can then be processed by accompanying software that presents detailed analytics to users.

One of the primary benefits of fitness trackers and their accompanying software is their ability to provide users with a comprehensive overview of their daily activity levels. By quantifying data that was once difficult to measure, these devices empower individuals to make informed decisions regarding their fitness goals. Whether it's increasing physical activity, managing weight, improving sleep, or monitoring heart health, fitness trackers can serve as effective personal health assistants that enable users to track their progress and adjust their routines as needed.

Despite their numerous advantages, fitness trackers and their software do have some limitations. One limitation concerns the interpretation and utilization of the collected data. In particular, while fitness tracker software presents users with comprehensive analytics, it is ultimately up to the individual to interpret and act upon the information. Without a proper understanding of how to use the data effectively, users may struggle to make meaningful changes to their routines.

Accordingly, it is desirable to provide improved techniques for managing workout routines using fitness trackers.

### SUMMARY

The described embodiments relate generally to techniques for managing workout profiles on computing devices. More particularly, the described embodiments provide techniques for generating workout objects from which workout profiles can be derived on client devices. The described embodiments also provide techniques for deriving workout profiles from workout objects on client devices.

One embodiment sets forth a method for generating workout objects from which workout profiles can be derived on client devices. According to some embodiments, the method can be implemented by a computing device that is communicatively coupled to a client device, and includes the steps of (1) receiving at least one workout parameter associated with a workout routine, (2) generating a workout object based on the workout routine, wherein the workout object is compatible with a fitness application installed on the client device, (3) providing the workout object to the fitness application to cause the fitness application to generate a workout profile that is based on the workout object and that is associated with at least one condition. (4) receiving, from the fitness application, progress information associated with the workout profile, and (5) generating at least one workout record associated with the workout object to reflect that the at least one condition is satisfied.

Another embodiment sets forth a method for deriving workout profiles from workout objects on client devices. According to some embodiments, the method can be implemented by a fitness application executing on a client device, and includes the steps of (1) receiving a workout object from an entity that is external to the fitness application. (2) generating, within the fitness application, a workout profile that is based on the workout object, wherein the workout profile is associated with at least one condition, (3) monitoring information gathered from at least one sensor that is communicatively coupled to the client device, and (4) in response to determining, based on the information, that the at least one condition is satisfied: causing at least one user interface to reflect that the at least one condition is satisfied.

Other embodiments include a non-transitory computer readable storage medium configured to store instructions that, when executed by a processor included in a computing device, cause the computing device to carry out the various steps of any of the foregoing methods. Further embodiments include a computing device that is configured to carry out the various steps of any of the foregoing methods.

Other aspects and advantages of the invention will become apparent from the following detailed description taken in conjunction with the accompanying drawings that illustrate, by way of example, the principles of the described embodiments.

### BRIEF DESCRIPTION OF THE DRAWINGS

The disclosure will be readily understood by the following detailed description in conjunction with the accompanying drawings, wherein like reference numerals designate like structural elements.

FIG. 1 illustrates a system diagram of a computing device that can be configured to perform the various techniques described herein, according to some embodiments.

FIG. 2 illustrates a sequence diagram of techniques for generating and implementing workout profiles, according to some embodiments.

FIGS. 3A-3C illustrate conceptual diagrams of user interfaces through which workout profiles can be generated and implemented, according to some embodiments.

FIG. 4 illustrates a method for generating workout objects from which workout profiles can be derived on client devices, according to some embodiments.

FIG. 5 illustrates a method for deriving workout profiles from workout objects on client devices, according to some embodiments.

FIG. 6 illustrates a detailed view of a computing device that can be used to implement the various techniques described herein, according to some embodiments.

### DETAILED DESCRIPTION

Representative applications of methods and apparatus according to the present application are described in this section. These examples are being provided solely to add context and aid in the understanding of the described embodiments. It will thus be apparent to one skilled in the art that the described embodiments may be practiced without some or all of these specific details. In other instances, well known process steps have not been described in detail in order to avoid unnecessarily obscuring the described embodiments. Other applications are possible, such that the following examples should not be taken as limiting.

In the following detailed description, references are made to the accompanying drawings, which form a part of the description, and in which are shown, by way of illustration, specific embodiments in accordance with the described embodiments. Although these embodiments are described in sufficient detail to enable one skilled in the art to practice the described embodiments, it is understood that these examples are not limiting; such that other embodiments may be used, and changes may be made without departing from the spirit and scope of the described embodiments.

The described embodiments relate generally to techniques for managing workout profiles on computing devices. More particularly, the described embodiments provide techniques for generating workout objects from which workout profiles can be derived on client devices. The described embodiments also provide techniques for deriving workout profiles from workout objects on client devices.

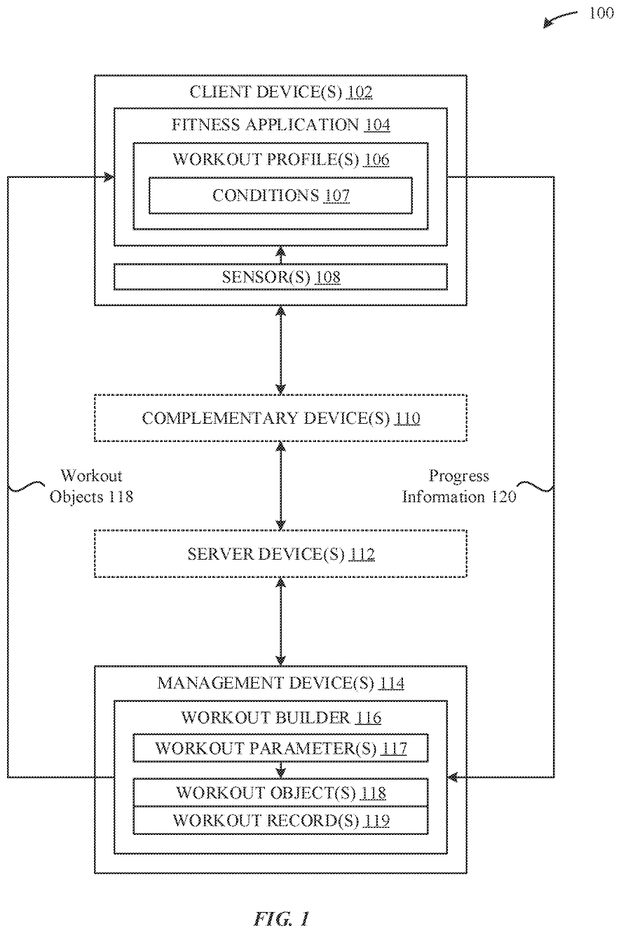

A more detailed discussion of these techniques is set forth below and described in conjunction with FIGS. 1-2, 3A-3C, and 4-6, which illustrate detailed diagrams of systems and methods that can be used to implement these techniques.

FIG. 1 illustrates a block diagram of different components of a system 100 that can be configured to implement the various techniques described herein, according to some embodiments. As shown in FIG. 1, the system 100 can include client devices 102, complementary devices 110, server devices 112, and management devices 114. According to some embodiments, each client device 102 can represent any computing device capable of tracking fitness activity, such as a smartphone device, a wearable device, a set-top box, a video game console, and so on. It is noted that the foregoing examples are not meant to be limiting, and that a given client device 102 can represent any form, type, etc., of computing device without departing from the scope of this disclosure.

According to some embodiments, each client device 102 can include sensors 108 that enable fitness-related information to be gathered by the client devices 102. For example, the sensors 108 can include timing sensors, accelerometer sensors, heart rate sensors, gyroscope sensors, Global Positioning System (GPS) sensors, altimeter sensors, thermometer sensors, Galvanic Skin Response (GSR) sensors, ambient light sensors, sleep sensors, ultraviolet (UV) light sensors, blood oxygen (SPO2) sensors, electrical heart rate sensors, bioimpedance sensors, cadence sensors, air pressure sensors, skin temperature sensors, respiration rate sensors, electrodermal activity (EDA) sensors, ultrasonic sensors, microphone sensors, camera/video sensors, muscle activity sensors, air quality sensors, blood pressure monitor sensors, humidity sensors, infrared sensors, magnetometer sensors, oxygen consumption (VO2 max) sensors, electrocardiogram (EKG) sensors electroencephalogram sensors, blood flow sensors, and the like. It is noted that the foregoing examples are not meant to be limiting, and that any form of sensor—including hardware-based and software-based sensors—can be included in the client device 102 without departing from the scope of this disclosure. It is also noted that the client devices 102 can be communicatively coupled with sensors that are not necessarily included within the client devices 102. For example, a given client device 102 can be configured to interface with other computing devices/sensors capable of gathering information and providing the information to the client device 102.

As shown in FIG. 1, each client device 102 can be configured to execute a fitness application 104, such as a fitness application that is native to an operating system (OS) executing on the client device 102, a fitness application associated with a third-party developer, and so on. According to some embodiments, the fitness application 104 can be configured to receive information from the sensors 108 to track fitness activity performed by a user of the client device 102. The fitness application 104 can also be configured to receive workout objects 118 from a workout builder 116 (described below in greater detail) and to generate workout profiles 106 based on the workout objects 118. According to some embodiments, a given workout profile 106 can enable specific fitness activities to be tracked. For example, a workout profile 106 can be related to swimming, and can involve tracking open-ended time, distance, heart rate, swim stroke, etc., metrics. In another example, a workout profile 106 can be related to cycling, and can involve tracking specific time, distance, heart rate, cadence, etc., goals. In yet another example, a workout profile 106 can encompass any number of activities at any level of granularity. For example, a workout profile 106 can be related to cycling, running, and swimming activities, where each activity includes respective time, distance, heart rate, and other fitness activity-relevant metrics/goals to be tracked/satisfied. It is noted that the foregoing examples are not meant to be limiting. and that a workout profile 106 can be configured to include any amount, type, etc., of information that enables the fitness application 104 to track fitness activity of any kind, at any level of granularity, without departing from the scope of this disclosure.

As shown in FIG. 1, each workout profile 106 can be associated with any number of conditions 107 that are tracked by the fitness application 104. According to some embodiments, the conditions 107 can include time-based conditions, location-based conditions, metric-based conditions, and so on. According to some embodiments, a given condition 107 can be specified within the workout object 118 (from which the workout profile 106 is derived). For example, a condition 107 included in the workout object 118 can require freestyle swimming for five minutes or three-hundred meters, whichever is satisfied first. Additionally, and according to some embodiments, a given condition 107 can be derived from the workout object 118, the workout profile 106, and/or any other information that is accessible to the client device 102. For example, the fitness application 104 can identify that a particular workout is to be performed at any recreational park that includes a variety of outdoor fitness equipment. In turn, the fitness application 104 can identify all satisfactory parks that are proximate to the user (e.g., within fifteen miles) and establish conditions 107 tied to geofence-based reminders associated with the parks. In this manner, the fitness application 104 can monitor the location of the client device 102 against the conditions 107 to determine whether a reminder should be presented to the user to engage in the workout when proximate to one of the aforementioned parks. It is noted that the foregoing examples are not meant to be limiting, and that the any number, type, form, etc., of conditions 107, derived from any information, at any level of granularity, can be implemented without departing from the scope of this disclosure.

As briefly discussed above, the fitness application 104 can be configured to receive workout objects 118 from which workout profiles 106 can be derived. According to some embodiments, the workout objects 118 can be generated/provided by a workout builder 116 that executes on a management device 114, a server device 112, a complementary device 110, and the like. Although not illustrated in FIG. 1, it is noted that a workout builder 116 can be executed on the client device 102 (or any other computing device not illustrated in FIG. 1/specifically described herein) without departing from the scope of this disclosure. According to some embodiments, the workout builder 116 can be configured to receive workout parameters 117, generate a workout object 118 based on the workout parameters 117, and then provide the workout object 118 to client devices 102. The workout parameters 117 can include, for example, a name for the workout, one or more fitness activities to be performed throughout the workout, specific goals to achieve for the fitness activities, specific goals to achieve for the workout, and so on. It is noted that the foregoing examples are not meant to be limiting, and that the workout parameters 117 can include any type, form, etc., of information, at any level of granularity, without departing from the scope of this disclosure. It is further noted that the workout builder 116 can modify the workout parameters 117, supplement the workout parameters 117, and so on, in conjunction with generating the workout objects 118, without departing from the scope of this disclosure.

As a brief aside, it is noted that the workout builder 116 can format the workout objects 118 using any approach that effectively enables the fitness applications 104 to interpret the workout objects 118 and generate workout profiles 106 based on the workout objects 118. For example, the workout object 118 can be formed using Extensible Markup Language (XML) approaches so that the workout objects 118 are stored in a “self-describing” format. In another example, the workout object 118 can be formed by serializing information into a binary file that can be interpreted by the fitness application 104. In yet another example, the workout object 118 can be formed by processing/storing the information into a proprietary file format that is understood by the fitness application 104. It is noted that the foregoing examples are not meant to be limiting, and that the workout objects 118 can be configured to incorporate any amount, type, format, etc., of information, at any level of granularity, without departing from the scope of this disclosure.

Additionally, and according to some embodiments, the workout builder 116 can be configured to receive, from client devices 102, progress information 120 associated with the workout profiles 106 (derived from the workout objects 118). In turn, the workout builder 116 can generate/manage workout records 119 that enable the workout builder 116 to track users' overall engagement with the workout profiles 106. For example, progress information 120 for a given workout profile 106 can indicate that a workout included in the workout profile 106 has been started, and can further indicate active metrics that are relevant to the workout. In this manner, the workout builder 116 can provide, e.g., via one or more user interfaces, an overview of the workout so that active/future adjustments to the workout profile 106 can be made where appropriate. Additionally, it is noted that authorization techniques can be implemented at any level of granularity without departing from the scope of this disclosure. For example, a client device 102 can be configured to display an authorization prompt prior to sending progress information 120 to a given entity, individual, etc., to ensure that a user of the client device 102 is aware of the progress information 120 being shared. The authorization prompt can indicate, for example, the type of information that is being shared, the recipients of the information, and so on. It is noted that the foregoing examples are not meant to be limiting, and that any approach that enables users of client devices 102 to control how progress information 120 is shared, at any level of granularity, can be implemented without departing from the scope of this disclosure.

It is noted that the complementary devices 110 can be optionally included within the system 100. A complementary device 110 can represent, for example, a smartphone, a tablet, a laptop computer, a desktop computer, etc., that is paired with one or more client devices 102 (e.g., fitness trackers). Such an arrangement can be beneficial, for example, when the complementary device 110 includes a larger screen that can aid the user in managing the client device(s) 102, when synchronization functionalities are desired, and so on. When complementary device(s) 110 are utilized by a given user, the information described herein—such as workout objects 118, workout profiles 106, the progress information 120, etc.—can be provided to the complementary devices 110 in any capacity. For example, a given workout object 118 can first be provided to a complementary device 110 of a client device 102. In turn, the complementary device 110 can generate a workout profile 106 based on the workout object 118, and then provide the workout profile 106 to the client device 102. In another example, a given workout object 118 can be first be provided to a client device 102. In turn, the client device 102 can generate a workout profile 106 based on the workout object 118, and then provide the workout profile 106 to the complementary device 110 and/or other client devices 102 that are associated with the complementary device 110. In yet another example, the complementary device 110 can execute the workout builder 116 and provide workout objects 118 and/or workout profiles 106 to the client devices 102. It is noted that the foregoing examples are not meant to be limiting, and that any distribution approach that involves (or omits) the complementary devices 110 can be utilized without departing from the scope of this disclosure.

It additionally is noted that the server devices 112 can be optionally included within the system 100. The server devices 112 can implement, for example, a cloud-based system with which the management devices 114, complementary devices 110, and/or the client devices 102 can be communicatively coupled. Such an arrangement can be beneficial, for example, when cloud-based workout object 118 generation, synchronization, etc., functionalities are desired. When server devices 112 are utilized, the information described herein—such as workout objects 118, workout profiles 106, progress information 120, etc.—can be provided to the server devices 112, complementary devices 110, and/or client devices 102 in any capacity. It is noted that the foregoing examples are not meant to be limiting, and that any distribution approach that involves (or omits) the server devices 112 can be utilized without departing from the scope of this disclosure.

It should be understood that the various components of the computing devices illustrated in FIG. 1 are presented at a high level in the interest of simplification. For example, although not illustrated in FIG. 1, it should be appreciated that the various computing devices can include common hardware/software components that enable the above-described software entities to be implemented. For example, each of the computing devices can include one or more processors that, in conjunction with one or more volatile memories (e.g., a dynamic random-access memory (DRAM)) and one or more storage devices (e.g., hard drives, solid-state drives (SSDs), etc.), enable the various software entities described herein to be executed. Moreover, each of the computing devices can include communications components that enable the computing devices to transmit information between one another.

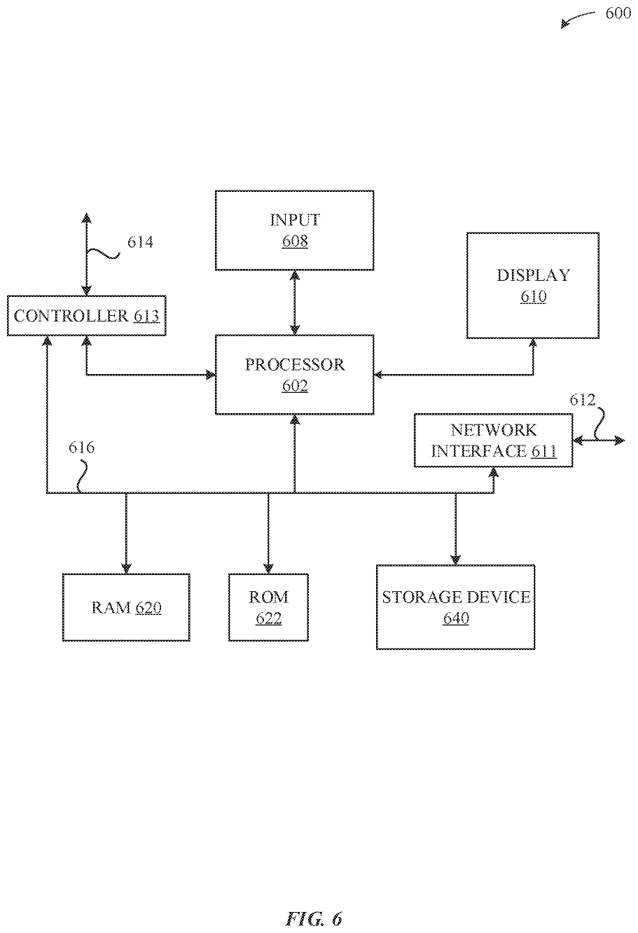

A more detailed explanation of these hardware components is provided below in conjunction with FIG. 6. It should additionally be understood that the computing devices can include additional entities that enable the implementation of the various techniques described herein without departing from the scope of this disclosure. It should additionally be understood that the entities described herein can be combined or split into additional entities without departing from the scope of this disclosure. It should further be understood that the various entities described herein can be implemented using software-based or hardware-based approaches without departing from the scope of this disclosure.

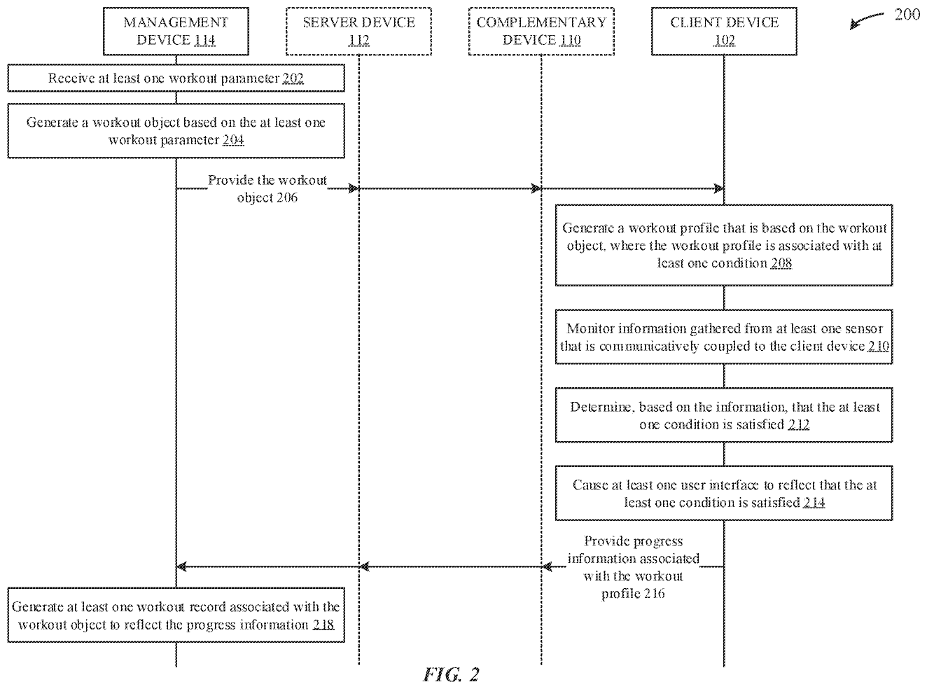

Accordingly, FIG. 1 provides an overview of the manner in which the system 100 can implement the various techniques described herein, according to some embodiments. A more detailed breakdown of the manner in which these techniques can be implemented will now be provided below in conjunction with FIGS. 2. 3A-3C, and 4-5.

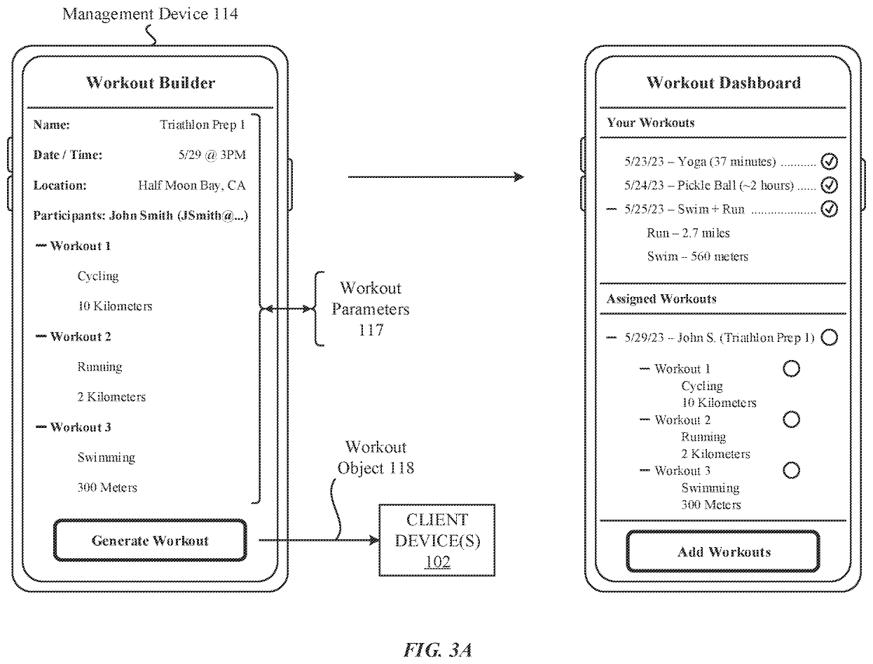

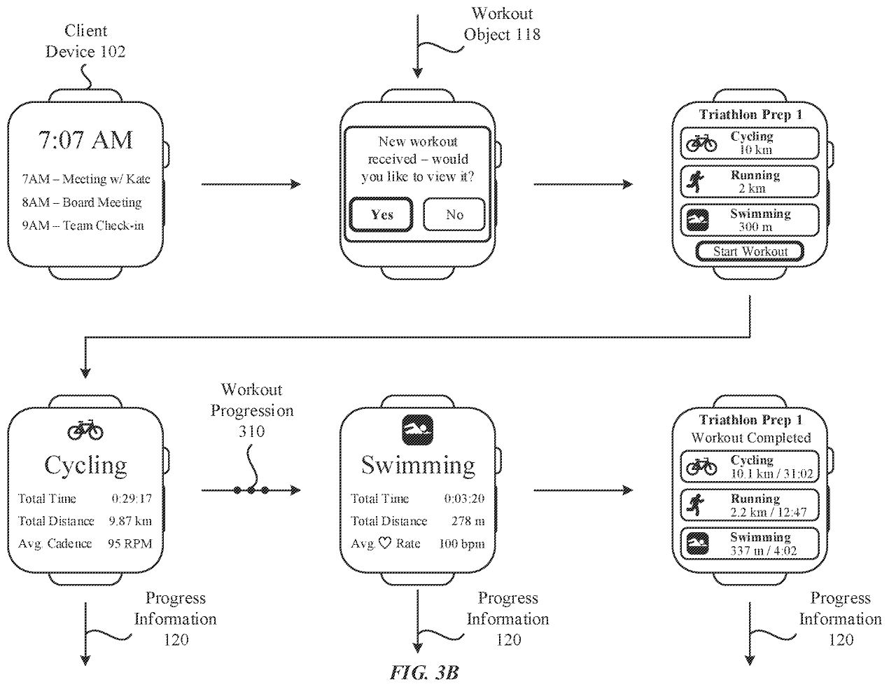

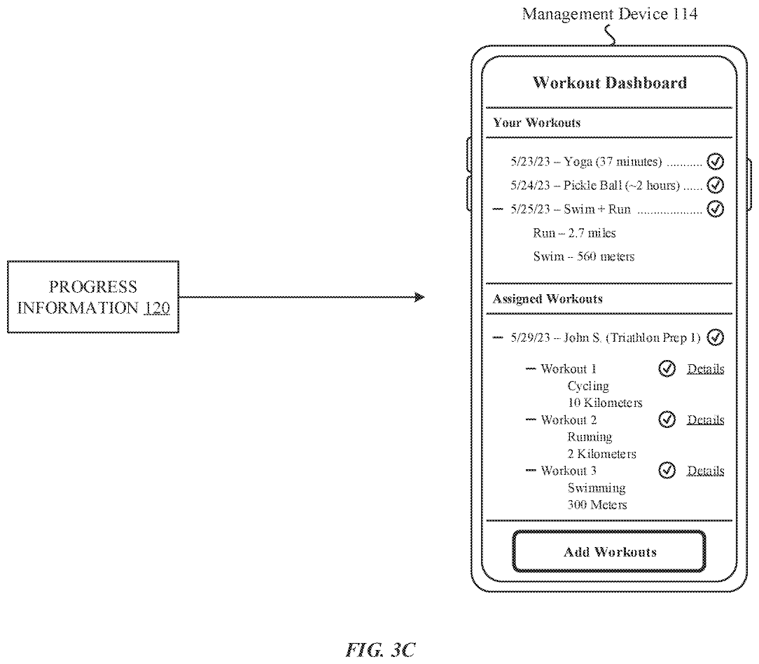

FIG. 2 illustrates a sequence diagram 200 of techniques for generating and implementing workout profiles 106, according to some embodiments. As shown in FIG. 2, the technique begins at step 202, where a management device 114 receives at least one workout parameter 117. As described herein, the management device 114 can execute a workout builder 116 that provides at least one user interface through which workout parameters 117 can be received, selected, provided, etc., such as the example user interfaces illustrated in FIG. 3A and described below in greater detail.

At step 204, the management device 114 generates a workout object 118 based on the at least one workout parameter 117 (e.g., as described above in conjunction with FIG. 1). Again, this can involve gathering the workout parameters 117 (modified/unmodified), information derived from the workout parameters 117, supplemental information, etc., and combining it into a workout object 118 (e.g., as a serialized file). For example, the workout builder 116 can interface with an Application Programming Interface (API) that provides at least one function that receives workout parameters 117 as inputs and generates a workout object 118 as an output. In turn, the workout builder 116 is positioned to provide the workout object 118 to one or more destination computing devices.

At step 206, the management device 114 provides the workout object 118 to one or more server devices 112 (where applicable), one or more complementary devices 110 (where applicable), and/or one or more client devices 102 (where applicable). According to some embodiments, the management device 114 can identify, e.g., based on the workout parameters 117, that the workout object 118 is directed to a particular user (e.g., a user account associated with the user). In turn, the management device 114 can identify a distribution path associated with the user account and route the workout object 118 based on the distribution path. For example, when the distribution path indicates that the user account is associated with only a single client device 102—and no complementary devices 110—then the management device 114 can route the workout object 118 to the client device 102 through one or more of the server devices 112 (and/or other computing devices not illustrated in FIGS. 1-2/described herein). In another example, when the distribution path indicates that the user account is associated with a complementary device 110 and a client device 102, then the management device 114 can route the workout object 118 to the complementary device 110 (which will then route the workout object 118 to the client device 102). Again, it is noted that the distribution paths discussed herein are not meant to be limiting, and that any distribution approach, involving any number of computing devices, communication mediums/channels, etc., can be utilized without departing from the scope of this disclosure.

At step 208, the client device 102 (e.g., a fitness application 104 executing thereon) generates a workout profile 106 that is based on the workout object 118, where the workout profile 106 is associated with at least one condition 107 (e.g., as described above in conjunction with FIG. 1). This step can involve, for example, the client device 102 accessing the workout object 118 via a filesystem that is implemented on the client device 102 and through which the workout object 118 is accessible. In this manner, the client device 102 can effectively import the workout object 118 as a workout profile 106, which can involve a variety of steps depending on how the workout object 118 is implemented. The steps can include, for example, extracting information from the workout object 118, modifying the information, incorporating supplemental information, and so on. In any case, at the conclusion of step 208, the fitness application 104 on the client device 102 is in possession of a workout profile 106 that corresponds to the workout object 118, where the workout profile 106 is associated with at least one condition 107. For example, the at least one condition 107 can be associated with a starting date/time that causes the client device 102 to display a reminder/prompt to activate the workout profile 106.

At step 210, the client device 102 monitors information gathered from at least one sensor 108 that is communicatively coupled to the client device 102 (e.g., as described above in conjunction with FIG. 1). This step can involve, for example, monitoring the current date/time, monitoring the location of the client device 102, monitoring whether a user of the client device 102 is engaging in fitness activity, monitoring whether the user has proactively selected the workout profile 106 (or another workout profile 106 managed by the fitness application 104), and so on. Again, it is noted that the foregoing examples are not meant to be limiting, and that the client device 102 can be configured to monitor any type, form, etc., of information, at any level of granularity, without departing from the scope of this disclosure. In this manner—and, as described below in conjunction with step 212—the client device 102 can utilize the information to identify instances where the client device 102 should perform one or more actions.

At step 212, the client device 102 determines, based on the information, that the at least one condition 107 is satisfied. Continuing with the example described above in conjunction with step 208, step 212 can involve analyzing whether the current date/time exceeds the starting date/time associated with the at least one condition 107. When the at least one condition 107 is satisfied, then the client device 102 can proceed to step 214. Otherwise, the client device 102 can continue to analyze information to determine whether the at least one condition 107 and/or other conditions 107 associated with other workout profiles 106 have been satisfied. As a brief aside, it is noted that the client device 102 can monitor the conditions 107 using any approach that is effective for achieving power savings. For example, event handlers can be created for the conditions 107 such that notifications when conditions 107 are satisfied are provided to the client device 102 (instead of the client device 102 proactively, periodically, and/or responsively reanalyzing the information to determine when the conditions 107 are satisfied).

At step 214, the client device 102 causes at least one user interface to reflect that the at least one condition 107 is satisfied. Continuing with the example described above in conjunction with steps 208 and 212, step 214 can involve the client device 102 displaying a notification, reminder, encouragement, etc., to activate the workout profile 106. It is noted that any approach can be utilized to effectively reflect (e.g., to a user of the client device 102) that the at least one condition 107 is satisfied, including audio-based notifications, haptic-based notifications, and the like.

At step 216, the client device 102 provides progress information 120 associated with the workout profile 106. As shown in FIG. 2, the progress information 120 can be transmitted in accordance with the distribution path discussed above in conjunction with step 206 of FIG. 2. which may include providing the progress information 120 to complementary device(s) 110, server device(s) 112, management device(s) 114, and/or other computing devices not illustrated in FIG. 2. In any case—and, continuing with the example described above in conjunction with steps 208, 212, and 214—step 216 can involve transmitting progress information 120 that indicates the client device 102 has presented a reminder to the user to activate the workout profile 106. The progress information 120 can also indicate, for example, that the workout profile 106 has been activated by the user, and include metrics associated with the user's fitness activity that are gathered by the fitness application 104 under the scope of the workout profile 106.

At step 218, the management device 114 generates at least one workout record 119 associated with the workout object 118 to reflect the progress information 120 (e.g., as described above in conjunction with FIG. 1). In this manner, the at least one workout record 119 can enable the workout builder 116 to track users' overall engagement with the workout profiles 106. For example, the workout builder 116 can output at least one user interface that enables a user to oversee the progress, efficacy, etc., of the workout profiles 106 generated using the workout builder 116, generated using other approaches and then shared with the workout builder 116, and so on. This information can be useful, for example, to trainers, coaches, doctors, physical therapists, etc., who have an interest in providing customized workout routines for individuals. For example, the progress information 120 can indicate whether a given individual is adhering to the prescribed workout routines, whether an overall difficulty of the routines is commensurate with the individual's current capacities, and so on.

Accordingly, the sequence diagram 200 of FIG. 2 illustrates the interactions between the various computing devices of FIG. 1 to effectively implement techniques for generating and implementing workout profiles 106, according to some embodiments. To provide additional context to the techniques described herein, FIGS. 3A-3C illustrate conceptual diagrams of user interfaces through which workout profiles 106 can be generated and implemented, according to some embodiments.

As shown in FIG. 3A, a management device 114—e.g., a workout builder 116 executing thereon—can output at least one user interface that enables a workout object 118 to be generated. The user interface can enable, for example, any type, form, etc. of information associated with a workout profile 106 to be selected, input, etc., as workout parameters 117, and can subsequently enable a workout object 118 to be generated based on the workout parameters 117. As shown in FIG. 3A, the example workout is named “Triathlon Prep 1”, is designated to begin on May 29^{th} at 3PM, and is designated to take place anywhere within Half Moon Bay in California. Additionally, the example workout is assigned to John Smith (e.g., via an email address). Additionally, the example workout includes three separate and distinct workouts: cycling (for ten kilometers), running (for two kilometers), and swimming (for three-hundred meters). It is noted that the example workout illustrated in FIG. 3A is merely exemplary and should not be construed as limiting. To the contrary, the example workout can incorporate any number, type, etc., of workout parameters 117, at any level of granularity, without departing from the scope of this disclosure.

As shown in FIG. 3A, a user of the management device 114 selects an option to generate a workout based on the workout parameters 117. In turn, the workout builder 116 generates a workout object 118 consistent with any of the workout object 118 generation techniques discussed herein. In turn, workout builder 116/management device 114 transmits the workout object 118 in accordance with the distribution path associated with John Smith (e.g., using any of the distribution techniques described herein). For example, the distribution path can indicate that a single client device 102 is possessed by John Smith, such that the management device 114 can transmit the workout object 118 to the client device 102 (e.g., using the Internet). In turn, the management device 114 can display a workout dashboard user interface that includes (1) information about workouts associated with the user of the management device 114, and (2) information about workouts assigned by the user of the management device 114 to other users (such as John Smith).

Additionally, and as shown in FIG. 3A, the workout dashboard can include checkbox UI elements that indicate whether the workout—and, where applicable, sub-components of a workout—have been completed. It is noted that the binary-valued (i.e., checkbox) UI elements illustrated in FIG. 3A are not meant to be limiting, and that the workout dashboard can incorporate any UI elements that effectively convey any amount, type, etc., of information, at any level of granularity, without departing from the scope of this disclosure. For example, the UI element associated with the header for John Smith's workout can take the form of a pie chart that indicates a total number of the sub-workouts that have been completed. For example, when Workout 1 and Workout 2 have been completed, the aforementioned UI element can be partially filled to indicate that two-thirds of John Smith's workout has been completed. Additionally, it is noted that the workout dashboard can include any type, form, etc., of information, at any level of granularity, to represent details associated with the progress of the workouts. For example, progress information 120 received from John Smith's client device 102 can be incorporated into the various workout displays to indicate active performance metrics as well as historical performance metrics within the scope of the workout. The workout dashboard can also enable assigned workouts to be modified prior to, during, etc., users' engagement with the workouts, by transmitting updated workout objects 118 to the appropriate computing devices.

FIG. 3B illustrates example user interfaces that can be displayed by John Smith's client device 102, according to some embodiments. As shown in FIG. 3B, the client device 102 can, prior to receiving the workout object 118 from the management device 114, display a lock/home screen that provides information such as the current time and upcoming calendar entries. In turn, when the client device 102 receives the workout object 118, the client device 102 can display a user interface that conveys to the user that a new workout has been received, and prompts the user about whether details of the new workout should be displayed. When the user opts to view the details, the client device 102 displays a user interface that includes the details of a workout profile 106 that is derived from the workout object 118 (e.g., using the techniques described herein). For example, as shown in FIG. 3B, the user interface includes the name of the workout, as well as information about the cycling, running, and swimming tasks described above in conjunction with FIG. 3A. The user interface also includes an option to start the workout. Alternatively, and as described herein, the user can simply dismiss the user interface. In turn, the client device 102 can alert the user when the starting date/time for conducting the workout has arrived (e.g., using the conditions 107 described herein).

As shown in FIG. 3B, when the user starts the workout, the client device 102 activates a user interface that is consistent with the structured workout defined by the workout profile 106. For example, the client device 102 displays a cycling fitness interface that displays a total time, a total distance, and an average cadence of a bike ride. In turn, the client device 102 can display a running fitness interface (not illustrated in FIG. 3B) and a swimming fitness interface that displays activity-relevant workout metrics, as indicated by the workout progression 310. Again, it is noted that the user interfaces illustrated in FIG. 3B are not meant to be limiting, and that the user interfaces can incorporate any form, type, etc., of information, at any level of granularity, without departing from the scope of this disclosure.

Additionally, and as shown in FIG. 3B, the client device 102 can output progress information 120 (e.g., periodically, continually, conditionally, etc.) to the management device 114. In turn, and as shown in FIG. 3C, the management device 114 can update the workout dashboard to reflect the progress information 120. For example, at the conclusion of the workout, the workout dashboard can incorporate the above-described checkbox UI elements to effectively convey to a user of the management device 114 that John Smith completed all aspects of the workout. Additionally, and as previously described herein, the workout dashboard can include options to view respective details associated with the aspects of the workout. For example, selecting “Details” for Workout 1 (cycling) can provoke the management device 114 to display one or more metrics gathered/calculated in conjunction with John Smith's cycling activity. Again, it is noted that the workout dashboard illustrated in FIG. 3C is not meant to be limiting, and that the workout dashboard can incorporate any amount, type, form, etc., of UI elements, to display any information at any level of granularity, without departing from the scope of this disclosure.

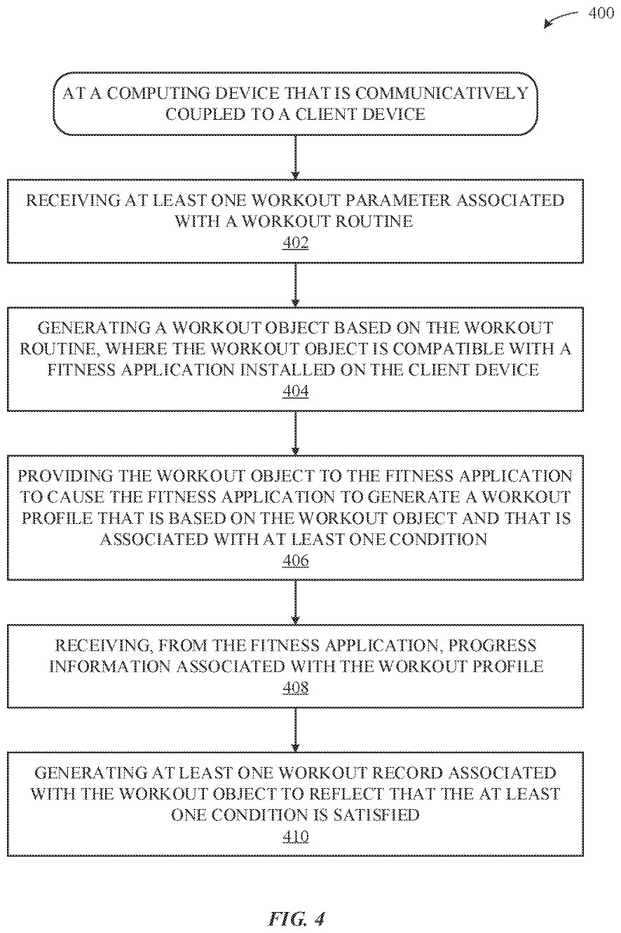

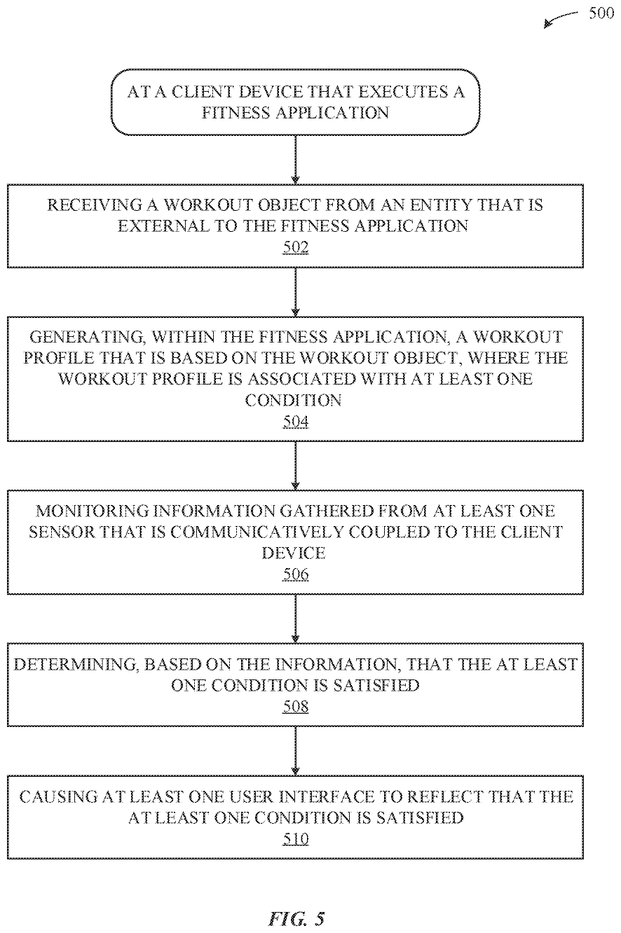

Accordingly, FIGS. 3A-3C illustrate conceptual diagrams of user interfaces through which workout profiles 106 can be generated and implemented, according to some embodiments. High-level breakdowns of the manners in which the entities discussed in conjunction with FIGS. 1-2 and 3A-3C can interact with one another will now be provided below in conjunction with FIGS. 4-5.

FIG. 4 illustrates a method 400 for generating workout objects from which workout profiles can be derived on client devices. According to some embodiments, the method can be implemented by a computing device that is communicatively coupled to the client device—such as a complementary device 110, a server device 112, a management device 114, and the like. As shown in FIG. 4, the method 400 begins at step 402, where the computing device receives at least one workout parameter associated with a workout routine (e.g., as described above in conjunction with FIGS. 1, 2, and 3A-3C).

At step 404, the computing device generates a workout object based on the workout routine, where the workout object is compatible with a fitness application installed on the client device (e.g., as also described above in conjunction with FIGS. 1, 2, and 3A-3C).

At step 406, the computing device provides the workout object to the fitness application to cause the fitness application to generate a workout profile that is based on the workout object and that is associated with at least one condition (e.g., as also described above in conjunction with FIGS. 1, 2, and 3A-3C).

At step 408, the computing device receives, from the fitness application, progress information associated with the workout profile (e.g., as also described above in conjunction with FIGS. 1, 2, and 3A-3C).

At step 410, the computing device generates at least one workout record associated with the workout object to reflect that the at least one condition is satisfied (e.g., as also described above in conjunction with FIGS. 1, 2, and 3A-3C).

FIG. 5 illustrates a method 500 for deriving workout profiles from workout objects on client devices 102. According to some embodiments, a fitness application 104 executing on the client device 102 can perform the steps illustrated in the method 500. As shown in FIG. 5, the method 500 begins at step 502, where the client device 102 receives a workout object from an entity that is external to the fitness application (e.g., as described above in conjunction with FIGS. 1, 2, and 3A-3C).

At step 504, the client device 102 generates, within the fitness application, a workout profile that is based on the workout object, where the workout profile is associated with at least one condition (e.g., as also described above in conjunction with FIGS. 1, 2, and 3A-3C).

At step 506, the client device 102 monitors information gathered from at least one sensor that is communicatively coupled to the client device (e.g., as also described above in conjunction with FIGS. 1, 2, and 3A-3C).

At step 508, the client device 102 determines, based on the information, that the at least one condition is satisfied.

At step 510, the client device 102 causes at least one user interface to reflect that the at least one condition is satisfied (e.g., as also described above in conjunction with FIGS. 1, 2, and 3A-3C).

FIG. 6 illustrates a detailed view of a computing device 600 that can be used to implement the various techniques described herein, according to some embodiments. In particular, the detailed view illustrates various components that can be included in the computing devices described in conjunction with FIG. 1. As shown in FIG. 6, the computing device 600 can include a processor 602 that represents a microprocessor or controller for controlling the overall operation of the computing device 600. The computing device 600 can also include a user input device 608 that allows a user of the computing device 600 to interact with the computing device 600. For example, the user input device 608 can take a variety of forms, such as a button, keypad, dial, touch screen, audio input interface, visual/image capture input interface, input in the form of sensor data, and so on. Still further, the computing device 600 can include a display 610 that can be controlled by the processor 602 (e.g., via a graphics component) to display information to the user. A data bus 616 can facilitate data transfer between at least a storage device 640, the processor 602. and a controller 613. The controller 613 can be used to interface with and control different equipment through an equipment control bus 614. The computing device 600 can also include a network/bus interface 611 that couples to a data link 612. In the case of a wireless connection, the network/bus interface 611 can include a wireless transceiver.

As noted above, the computing device 600 also includes the storage device 640, which can comprise a single disk or a collection of disks (e.g., hard drives). In some embodiments, storage device 640 can include flash memory, semiconductor (solid-state) memory or the like. The computing device 600 can also include a Random-Access Memory (RAM) 620 and a Read-Only Memory (ROM) 622. The ROM 622 can store programs, utilities, or processes to be executed in a non-volatile manner. The RAM 620 can provide volatile data storage, and stores instructions related to the operation of applications executing on the computing device 600.

The various aspects, embodiments, implementations, or features of the described embodiments can be used separately or in any combination. Various aspects of the described embodiments can be implemented by software, hardware or a combination of hardware and software. The described embodiments can also be embodied as computer readable code on a computer readable medium. The computer readable medium is any data storage device that can store data that can be read by a computer system. Examples of the computer readable medium include read-only memory, random-access memory, CD-ROMs, DVDs, magnetic tape, hard disk drives, solid state drives, and optical data storage devices. The computer readable medium can also be distributed over network-coupled computer systems so that the computer readable code is stored and executed in a distributed fashion.

The foregoing description, for purposes of explanation, used specific nomenclature to provide a thorough understanding of the described embodiments. However, it will be apparent to one skilled in the art that the specific details are not required in order to practice the described embodiments. Thus, the foregoing descriptions of specific embodiments are presented for purposes of illustration and description. They are not intended to be exhaustive or to limit the described embodiments to the precise forms disclosed. It will be apparent to one of ordinary skill in the art that many modifications and variations are possible in view of the above teachings.

The terms “a,” “an,” “the,” and “said” as used herein in connection with any type of processing component configured to perform various functions may refer to one processing component configured to perform each and every function, or a plurality of processing components collectively configured to perform the various functions. By way of example, “A processor” configured to perform actions A, B, and C may refer to one or more processors configured to perform actions A, B, and C. In addition, “A processor” configured to perform actions A, B, and C may also refer to a first processor configured to perform actions A and B, and a second processor configured to perform action C. Further, “A processor” configured to perform actions A, B, and C may also refer to a first processor configured to perform action A, a second processor configured to perform action B, and a third processor configured to perform action C.

In addition, in methods described herein where one or more steps are contingent upon one or more conditions having been met, it should be understood that the described method can be repeated in multiple repetitions so that over the course of the repetitions all of the conditions upon which steps in the method are contingent have been met in different repetitions of the method. For example, if a method requires performing a first step if a condition is satisfied, and a second step if the condition is not satisfied, then a person of ordinary skill would appreciate that the claimed steps are repeated until the condition has been both satisfied and not satisfied, in no particular order. Thus, a method described with one or more steps that are contingent upon one or more conditions having been met could be rewritten as a method that is repeated until each of the conditions described in the method has been met. This, however, is not required of system or computer readable medium claims where the system or computer readable medium contains instructions for performing the contingent operations based on the satisfaction of the corresponding one or more conditions and thus is capable of determining whether the contingency has or has not been satisfied without explicitly repeating steps of a method until all of the conditions upon which steps in the method are contingent have been met. A person having ordinary skill in the art would also understand that, similar to a method with contingent steps, a system or computer readable storage medium can repeat the steps of a method as many times as are needed to ensure that all of the contingent steps have been performed.

As described herein, one aspect of the present technology is the gathering and use of data available from various sources to improve user experiences. The present disclosure contemplates that in some instances, this gathered data may include personal information data that uniquely identifies or can be used to contact or locate a specific person. Such personal information data can include demographics data, location-based data, telephone numbers, email addresses, home addresses, data or records relating to a user's health or level of fitness (e.g., vital signs measurements, medication information, exercise information), date of birth, smart home activity, or any other identifying or personal information. The present disclosure recognizes that the use of such personal information data, in the present technology, can be used to the benefit of users.

The present disclosure contemplates that the entities responsible for the collection, analysis, disclosure, transfer, storage, or other use of such personal information data will comply with well-established privacy policies and/or privacy practices. In particular, such entities should implement and consistently use privacy policies and practices that are generally recognized as meeting or exceeding industry or governmental requirements for maintaining personal information data private and secure. Such policies should be easily accessible by users, and should be updated as the collection and/or use of data changes. Personal information from users should be collected for legitimate and reasonable uses of the entity and not shared or sold outside of those legitimate uses. Further, such collection/sharing should occur after receiving the informed consent of the users. Additionally, such entities should consider taking any needed steps for safeguarding and securing access to such personal information data and ensuring that others with access to the personal information data adhere to their privacy policies and procedures. Further, such entities can subject themselves to evaluation by third parties to certify their adherence to widely accepted privacy policies and practices. In addition, policies and practices should be adapted for the particular types of personal information data being collected and/or accessed and adapted to applicable laws and standards, including jurisdiction-specific considerations. For instance, in the US, collection of or access to certain health data may be governed by federal and/or state laws, such as the Health Insurance Portability and Accountability Act (HIPAA); whereas health data in other countries may be subject to other regulations and policies and should be handled accordingly. Hence different privacy practices should be maintained for different personal data types in each country.

Despite the foregoing, the present disclosure also contemplates embodiments in which users selectively block the use of, or access to, personal information data. That is, the present disclosure contemplates that hardware and/or software elements can be provided to prevent or block access to such personal information data. For example, the present technology can be configured to allow users to select to “opt in” or “opt out” of participation in the collection of personal information data during registration for services or anytime thereafter. In another example, users can select to provide only certain types of data that contribute to the techniques described herein. In addition to providing “opt in” and “opt out” options, the present disclosure contemplates providing notifications relating to the access or use of personal information. For instance, a user may be notified that their personal information data may be accessed and then reminded again just before personal information data is accessed.

Moreover, it is the intent of the present disclosure that personal information data should be managed and handled in a way to minimize risks of unintentional or unauthorized access or use. Risk can be minimized by limiting the collection of data and deleting data once it is no longer needed. In addition, and when applicable, including in certain health related applications, data de-identification can be used to protect a user's privacy. De-identification may be facilitated, when appropriate, by removing specific identifiers (e.g., date of birth, etc.), controlling the amount or specificity of data stored (e.g., collecting location data a city level rather than at an address level), controlling how data is stored (e.g., aggregating data across users), and/or other methods.

Therefore, although the present disclosure broadly covers use of personal information data to implement one or more various disclosed embodiments, the present disclosure also contemplates that the various embodiments can also be implemented without the need for accessing such personal information data. That is, the various embodiments of the present technology are not rendered inoperable due to the lack of all or a portion of such personal information data.

What is claimed is:

1. A method for generating workout objects from which workout profiles can be derived on client devices, the method comprising, by a computing device that is communicatively coupled to a client device:
receiving at least one workout parameter associated with a workout routine;
generating a workout object based on the workout routine, wherein the workout object is compatible with a fitness application installed on the client device;
providing the workout object to the fitness application to cause the fitness application to generate a workout profile that is based on the workout object and that is associated with at least one condition;
receiving, from the fitness application, progress information associated with the workout profile; and
generating at least one workout record associated with the workout object to reflect that the at least one condition is satisfied.

2. The method of claim 1, further comprising:
causing at least one user interface to reflect that the at least one condition is satisfied.

3. The method of claim 1, wherein the at least one workout parameter comprises workout parameters for two separate and distinct types of workouts.

4. The method of claim 1, further comprising, prior to receiving the at least one workout parameter:
providing at least one user interface that enables the at least one workout parameter to be received.

5. The method of claim 1, wherein generating the workout object based on the workout routine comprises:
providing the at least one workout parameter to an Application Programming Interface (API) that is accessible to the computing device; and
receiving the workout object from the API.

6. The method of claim 1, wherein the computing device comprises:
a complementary device with which the client device is communicably paired, or
a server device with which the client device is communicably paired, wherein the server device receives the workout object from a management device.

7. A method for deriving workout profiles from workout objects on client devices, the method comprising, by a fitness application executing on a client device:
receiving a workout object from an entity that is external to the fitness application;
generating, within the fitness application, a workout profile that is based on the workout object, wherein the workout profile is associated with at least one condition;
monitoring information gathered from at least one sensor that is communicatively coupled to the client device; and
in response to determining, based on the information, that the at least one condition is satisfied:
causing at least one user interface to reflect that the at least one condition is satisfied.

8. The method of claim 7, wherein the fitness application is a native application of an operating system (OS) executing on the client device.

9. The method of claim 7, wherein:
the information comprises a current date/time;
the at least one condition is satisfied when the current date/time matches and/or exceeds a scheduled date/time associated with the workout profile; and
causing the at least one user interface to reflect that the at least one condition is satisfied comprises displaying a prompt to activate the workout profile.

10. The method of claim 7, wherein:
the information comprises at least one workout metric;
the at least one condition is satisfied when the at least one workout metric satisfies at least one workout parameter associated with the workout profile; and
causing the at least one user interface to reflect that the at least one condition is satisfied comprises displaying at least one notification that the at least one workout parameter is satisfied.

11. The method of claim 10, wherein the method further comprises:
providing, to the entity, progress information associated with the workout profile.

12. The method of claim 11, wherein the progress information causes the entity to update at least one workout record associated with the workout object to reflect the progress information.

13. The method of claim 7, wherein the entity comprises:
a complementary device with which the client device is communicably paired, or
a server device with which the client device is communicably paired, wherein the server device receives the workout object from a management device.

14. A non-transitory computer readable storage medium configured to store instructions that, when executed by at least one processor included in a client device, cause the client device to execute a fitness application that derives workout profiles from workout objects, by carrying out steps that include:
receiving a workout object from an entity that is external to the fitness application;
generating, within the fitness application, a workout profile that is based on the workout object, wherein the workout profile is associated with at least one condition;
monitoring information gathered from at least one sensor that is communicatively coupled to the client device; and
in response to determining, based on the information, that the at least one condition is satisfied:
causing at least one user interface to reflect that the at least one condition is satisfied.

15. The non-transitory computer readable storage medium of claim 14, wherein the fitness application is a native application of an operating system (OS) executing on the client device.

16. The non-transitory computer readable storage medium of claim 14, wherein:
the information comprises a current date/time;
the at least one condition is satisfied when the current date/time matches and/or exceeds a scheduled date/time associated with the workout profile; and
causing the at least one user interface to reflect that the at least one condition is satisfied comprises displaying a prompt to activate the workout profile.

17. The non-transitory computer readable storage medium of claim 14, wherein:
the information comprises at least one workout metric;
the at least one condition is satisfied when the at least one workout metric satisfies at least one workout parameter associated with the workout profile; and
causing the at least one user interface to reflect that the at least one condition is satisfied comprises displaying at least one notification that the at least one workout parameter is satisfied.

18. The non-transitory computer readable storage medium of claim 17, wherein the steps further include:
providing, to the entity, progress information associated with the workout profile.

19. The non-transitory computer readable storage medium of claim 18, wherein progress information causes the entity to update at least one workout record associated with the workout object to reflect the progress information.

20. The non-transitory computer readable storage medium of claim 14, wherein the entity comprises:
a complementary device with which the client device is communicably paired, or
a server device with which the client device is communicably paired, wherein the server device receives the workout object from a management device.
